(19) United States
(12) Patent Application Publication
(10) Pub. No.: US 2006/0198854 A1
(43) Pub. Date: Sep. 7, 2006

(54) Vector platforms derived from the alphavirus vaccines

(76) Inventor: Peter Pushko, Frederick, MD (US)

Correspondence Address: BUCHANAN INGERSOLL PC, POST OFFICE BOX 1404, ALEXANDRIA, VA, 22313-1404, US

(21) Appl. No.: 11/319,214

(22) Filed: Dec. 28, 2005

Related U.S. Application Data
(60) Provisional application No. 60/639,346, filed on Dec. 28, 2004.

(51) Int. Cl. C07H 21/04; A61K 39/12

(52) U.S. Cl. 424/218.1; 536/23.72

(57) ABSTRACT

Nucleic acid molecules and vector platforms derived from human virus vaccines, for example the alphavirus vaccines, including the TC-83 human vaccine, are disclosed. These vector platforms can provide for expression of a heterologous protein or nucleic acid in animal or human cells. In preferred embodiments, the nucleic acid molecules and vector platforms can be safely used to make and administer vaccines or gene therapies.

This application claims the benefit of U.S. Provisional Application No. 60/639,346, filed Dec. 28, 2004, which is incorporated herein by reference in its entirety. Subject matter described herein was also described in Disclosure Document No. 556,822 filed in the United States Patent Office on Aug. 11, 2004, which is incorporated herein by reference in its entirety.

The inventor received material related to this invention from the U.S. government under an agreement pursuant to 15 U.S.C. §3710a, accordingly the U.S. government may have certain rights in the invention.

### BACKGROUND OF THE INVENTION

1. Field of the Invention

The invention relates to gene vectors made from viral vaccines and systems and methods for making and using such vectors.

2. Description of the Related Art

Vector platforms have been previously developed from various alphaviruses. See, e.g., U.S. Pat. Nos. 6,376,236 and 5,843,723. Vaccine candidates against several diseases have been developed using these platforms. For example, Venezuelan equine encephalitis virus (VEE) has been used a vector platform. (Pushko et al., 1997; U.S. Pat. Nos. 6,541,010; 5,792,462; 6,531,135). In pre-clinical studies, vaccine candidates made using the VEE vector platform successfully protected animals from various diseases, including Ebola hemorrhagic fever (Pushko et al., 2000, 2001), Lassa fever (Pushko et al., 2001), influenza (Pushko et al.; 1997), Marburg virus infection (Hevey et al., 1998; U.S. Pat. No. 6,517,842), Staphylococcus intoxication (U.S. Pat. No. 6,632,640), anthrax (U.S. Pat. No. 6,770,479), cancer (Nelson et al., 2003), and other illnesses. However, alphavirus vector platforms including those derived from VEE, can possess inherent safety problems.

### SUMMARY

Safe gene vector platforms can be derived from attenuated alphavirus vaccines. Preferred embodiments include a RNA molecule in the form of a replicon comprising an alphavirus 5′ untranslated region, an alphavirus non-structural gene region, and an alphavirus 3′ untranslated region, and further comprising a RNA-dependent RNA polymerase promoter region operably coupled to a heterologous nucleic acid sequence upstream of the 3′ untranslated region, wherein one or more attenuating mutations are present in one or more of said alphavirus regions. A replicon may consist essentially of these elements, together with sequence elements such as those that are routinely used in the art for the convenience of the practitioner in manipulating or purifying the nucleic acid molecule. Such a replicon may be deleted of a structural gene region. In preferred embodiments, the attenuating mutations or entire regions of the RNA molecule are mutations or regions present in the TC-83 VEE alphavirus vaccine (GenBank Accession No. L01443). Particularly preferred mutations include the substitution of an adenosine (A) in the position corresponding to nucleotide 3 of the TC-83 VEE genome as described in GenBank Accession No. L01443 and substitution of a guanidine (G) in the position corresponding to nucleotide 8922 of the TC-83 VEE genome as described in GenBank Accession No. L01443.

A helper RNA molecule may by used to package these RNA molecules into virus particles in a host cell. A helper RNA molecule can comprise an alphavirus genome from which the non-structural gene region has been deleted. Preferred examples include, an RNA molecule comprising an isolated RNA polymerase region operably coupled to an alphavirus structural gene region. In a preferred embodiment, a helper RNA molecule can consist essentially of a RNA polymerase region operably coupled to an alphavirus structural gene region, and may also include 3′ and 5′ untranslated regions. In preferred embodiments, the structural gene region comprises one or more attenuating mutations, for example one or more mutations found in the structural region of the TC-83 VEE genome.

Alternatively, a RNA molecule can comprise an alphavirus 5′ untranslated region, an alphavirus non-structural gene region, a first RNA-dependent RNA polymerase promoter region, an alphavirus structural gene region, and an alphavirus 3′ untranslated region, wherein one or more attenuating mutations are present in one or more of these regions, and the RNA molecule further comprising a RNA-dependent RNA polymerase promoter region operably coupled to a heterologous nucleic acid sequence upstream of the 3′ untranslated region. As above, the attenuating mutations or entire regions of the RNA molecule are preferably mutations or regions present in the TC-83 VEE alphavirus vaccine (GenBank Accession No. L01443). Particularly preferred mutations include the substitution of an adenosine (A) in the position corresponding to nucleotide 3 of the TC-83 VEE genome as described in GenBank Accession No. L01443 and substitution of a guanidine (G) in the position corresponding to nucleotide 8922 of the TC-83 VEE genome as described in GenBank Accession No. L01443. Such a RNA molecule can comprise an attenuated alphavirus replicon comprising a non-structural alphavirus gene region having attenuating mutations and a structural gene region having attenuating mutations so that the molecule is capable of self replication and packaging, yet remains safe due to the attenuating mutations in both non-structural regions and structural regions.

A system or platform can include vector molecules and helper molecules or host cells comprising nucleotide sequences comprising or encoding vector molecules and/or helper molecules as described above. In alternative embodiments, a system may include a multipart helper comprising a plurality of helper RNA molecules, each of which comprise a different portion of an alphavirus structural gene region, i.e. each helper being capable of providing for expression of a different structural protein. For example, a bipartite helper comprises two RNA molecules, each comprising a different portion of an alphavirus structural gene region.

In addition, DNA molecules can comprise one or more sequence elements encoding a vector or replicon and/or one or more helper RNA molecules as described above, preferably operably linked to DNA regulatory elements including but not limited to DNA dependant RNA prolymerase promoter regions.

RNA vector molecules may be packaged or encapsidated to form vector particles. Packaging may occur in any permissive cell line or in a host organism. Methods of making vector particles comprise introducing a nucleic acid sequence encoding a vector, or RNA replicon sequences, into a host cell, for example by transfection or electroporation. Cells can comprise one or more helper nucleotide sequences as plasmids or stably expressed transgenes capable of expressing alphavirus structural proteins. Such host cells can be incubated in any appropriate media so as to permit the cells to produce packaged viral particles and the particles are recovered from the cells or media.

Methods of using the vectors can comprise administering vector particles and/or nucleic acids encoding vectors to a host animal or human in vivo, or introducing vector particles or nucleic acid sequences into host cells ex vivo. In preferred embodiments a method of using vectors can comprise administering DNA or RNA vectors, which may be combined with liposomes or similar transfection or targeting agents, directly to an animal or human.

### BRIEF DESCRIPTION OF THE DRAWINGS

FIG. 1 illustrates self-replicating (replicon) and helper RNAs derived from TC-83 or other alphavirus live attenuated vaccines. Locations of two attenuating mutations within the TC-83 vaccine genome are indicated with stars. Mutation within the 5′ terminus of the TC-83 genome is present within the replicon molecules (see also FIG. 4). Locations of two attenuating mutations in vaccine strain V3526 are shown with triangles. Regions that can be deleted in replicon and helper molecules are indicated with broken lines. The untranslated regions (UTR) are important for replication of the molecules.

FIG. 2 illustrates vector platforms incapable of self propagation that are derived from alphavirus vaccines. The filled arrow indicates a DNA-dependent RNA polymerase promoter (such as a CMV promoter/enhancer sequence). The open arrows indicate an RNA-dependent RNA polymerase promoter (such as the 26S promoter). An “x” indicates the location of a heterologous gene. A star indicates a location of an attenuating mutation at nucleotide position 3 in the TC-83 vaccine genome.

FIG. 3 shows a schematic diagram of one type of alphavirus-like replicon particle containing a TC-83 replicon expressing foreign gene. Virus-like replicon particle (VRP) envelope is shown as an octagon, capsid is shown as a circle inside the octagon. An open rectangle illustrates a non-structural gene region derived from a live attenuated alphavirus. A solid vertical arrow indicates an attenuating mutation in the encapsidated replicon. An open arrow indicates a subgenomic 26S promoter. Solid rectangle indicates the location of a heterologous gene sequence for vaccine or therapy. Right panel, cryoelectron microscopy image reconstruction of alphavirus particle.

FIG. 4 illustrates an embodiment of a vector platform or system derived from TC-83 vaccine. A bipartite helper is shown. Solid vertical arrows indicate the locations of two attenuating mutations that have been identified (Kinney et al., 1993). The replicon RNA contains TC-83 non-structural genes and a heterologous gene sequence, i.e. for vaccine- or/and therapy-relevant gene expression. Replicon RNA can be encapsidated into alphavirus-like replicon particles using bipartite packaging helpers encoding TC-83 structural proteins (i.e. capsid and glycoproteins PE2 and E1). Open arrows indicate subgenomic 26S promoter sequences.

FIG. 5 shows the nucleotide sequence of the TC-83 vaccine genome as described in GenBank accession number L01443.

FIG. 6 shows an alignment of 5′ untranslated termini of Venezuelan equine encephalitis (VEE) virus genomes from GenBank. GenBank accession numbers are shown on the right. The TC-83 sequence is on the top (accession number L01443). A unique mutation in position 3 of the TC-83 sequence is underlined and indicated with a solid arrow.

FIG. 7 shows an alignment of VEE E2 gene fragment sequences in the vicinity of E2-120. Sequences are from GenBank, accession numbers are shown on the right, TC-83 sequence is on the top (accession number L01443). A unique mutation in the TC-83 sequence is underlined and indicated with a solid arrow.

FIG. 8 illustrates vector platforms capable of limited or continuous propagation that are derived from the alphavirus vaccines. A filled arrow indicates a DNA-dependent RNA polymerase promoter (such as a CMV promoter/enhancer). Open arrows indicate RNA-dependent RNA polymerase promoters (such as the 26S promoter). An “x” indicates a heterologous gene. An “sp” indicates a structural protein region comprising structural protein genes. Stars indicate the location of an attenuating mutation at nucleotide positions 3 and 8922 of the TC-83 vaccine genome. In comparison to FIG. 2, particles generated in the cell in vitro or in vivo are capable of infecting other cells, which can lead to expression of a vaccine or therapeutic product in many cells thus augmenting production of the product (e.g., in vitro) or vaccination and/or therapeutic effect in vivo.

FIG. 9 (A-C) illustrates an overview of vectors and vector platforms derived from alphavirus vaccines. Heterologous genes, which can encode a prophylactic or therapeutically relevant product, are indicated with “x”. Structural regions are inditaced by black boxes. (A) Vectors and vector platforms capable of propagation. (B) Vectors and vector platforms capable of limited propagation. (C) Vectors and vector platforms capable of propagation only under special conditions (concentration, encapsulation, etc.). In such vectors and vector platforms derived from alphavirus vaccines, DNA molecules can be introduced directly in vivo or in cultured cells, in order to generate RNA molecules (e.g., replicons) capable of directing the expression of prophylactic or therapeutically relevant product(s). Alternatively, RNA molecules are generated by in vitro transcription from DNA molecules. In the latter case, RNA molecules can be introduced directly in vivo or in cultured cells, in order to generate replicated RNA molecules that express prophylactic or therapeutically relevant product(s). Alternatively, replicon-containing virus-like particles are generated in cultured cells or in vivo host cells that replicate and package the RNA molecules. Such particles can be used to introduce the RNA molecules capable of directing expression of prophylactic or therapeutically relevant products into cultured cells or host cell in vivo

FIG. 10. Transfection of CHO cells using pRM03 plasmid DNA and transfection reagent (Fugene 6, Roche Applied Sciences), imaged by indirect immunofluorescent assay (IFA). Arrows indicate CHO cells expressing influenza hemagglutinin protein. For detection of influenza protein, rabbit antiserum to influenza virus was used followed by rhodamine-conjugated goat antiserum to rabbit IgG.

### DETAILED DESCRIPTION OF PREFERRED EMBODIMENTS OF THE INVENTION

Alphavirus vector platforms including those derived from the Venezuelan equine encephalitis virus (VEE), possess inherent safety problems that make administration into people for vaccine or therapy purposes problematic. Alphaviruses can cause illnesses in people ranging from mild symptoms to severe disease and death. For example, VEE virus is a human pathogen capable of causing lethal encephalitis in humans. According to the Centers for Disease Control (Atlanta, Ga.), VEE virus is a Category B Select Agent and a human pathogen assigned to one of the highest degrees of biosafety containment, BSL-3. Any research in the U.S. involving VEE virus requires BSL-3 biocontainment facilities and a special immunization program for the personnel involved. Live attenuated TC-83 vaccine is used in the U.S. to immunize personnel at risk of infection with VEE virus.

Precautions including BSL-3 containment and a special immunization program are designed to prevent life-threatening illnesses in laboratory workers, and prevent VEE virus from the escaping into environment, where the virus can quickly spread to people and animals via mosquitoes. Such safety concerns surround research, development, manufacturing, and application of vector systems derived from the VEE virus as well as from the other alphaviruses. Previous VEE-based vectors, vaccines, and/or therapies could pose significant safety risks if used in people.

The main component of alphavirus vector platforms is an RNA molecule that includes approximately two-thirds or more of the alphavirus RNA genome that is capable of self replication (a “replicon”) and a heterologous gene. As used herein, a “replicon” refers to an RNA molecule capable of self replication. An alphavirus derived replicon generally comprises at least a 5′ untranslated region, the non-structural region, which is the region of the alphavirus that encodes enzymes capable of replicating the RNA molecule (“replicase”), and a 3′ untranslated region. A replicon can also comprise a heterologous gene region upstream of the 3′ untranslated region and may also comprise a region encoding the structural genes of the alphavirus. Alternatively, the structural gene region may be provided in trans to package replicon particles.

A self-replicating RNA vector derived from VEE virus, a human pathogen, represents a safety concern, especially when the full-length VEE RNA genome is used as a vector (Davis et al., 1996) For increased safety, the replicon may contain only the approximately two-thirds of the VEE genome required for self-replication, but omit some or all of the structural genes. In this case, alphavirus vector platforms can utilize helper molecules that encode the structural proteins of the alphavirus particle, including nucleocapsid and spike glycoproteins. In such vector systems, the structural proteins encapsidate the replicon RNA molecules into the propagation-defective alphavirus-like replicon particles. Such particles represent highly efficient vectors for delivery of replicon RNA, including a heterologous gene, into eukaryotic cells.

However, regeneration of the full-length infectious VEE RNA is possible (Pushko et al., 1997). Infectious alphavirus can regenerate through recombination between the replicon and helper nucleic acid molecules. This can result in regeneration of live, infectious alphavirus via recombination between the replicon and helper molecules during vector manufacturing. The presence of such regenerated virus can lead to contamination of vector preparations with infectious, life-threatening pathogens, which is not acceptable for human vaccines or therapies.

In order to reduce safety risks, a VEE vector was derived from a mutated cDNA clone that encoded a VEE virus that was attenuated in mice (Davis et al., 1996; Pushko et al., 1997). However, there is no data suggesting that such mutant viruses are similarly attenuated in humans. In some cases, mutant VEE viruses have been shown to be highly attenuated in mice but not in other animals such as hamsters (Davis et al., 1991). Further, in the VEE vectors developed so far, attenuating mutations have been located only in the structural genes. Therefore, the VEE replicon vector itself had no attenuating mutations at all and comprised at least two thirds, or even the full-length wild-type, pathogenic VEE virus RNA. The replicon RNA containing two thirds of the VEE genome, can undergo recombination with the helper, which would result in the regeneration of full-length, infectious VEE virus. In order to further reduce possibilities for regeneration of live VEE via recombination between the replicon and helper, a bipartite helper can be used (Pushko et al., 1997; U.S. Pat. No. 5,792,462).

Other approaches also have been pursued to improve safety. Other researchers developed vaccine platforms from less pathogenic alphaviruses, such as Sindbis virus and Semliki Forest virus (Polo et al., 1999; Smerdou et al., 1999). However, these vaccine platforms lack some useful features of the VEE vaccine vector platform, and have their own safety problems. For example, a lethal human case was reported for Semliki Forest virus. (Willems et al., 1979). Others have made chimeric vaccine platform by using components from Sindbis, VEE, and Semliki Forest viruses. Additionally, attempts were made to improve safety by using packaging cell lines and DNA constructs. However, all these approaches suffer from a lack of available safety data regarding the resulting alphavirus vectors and the alphaviruses that were used for the development of these alphavirus vectors. This makes the use of the previous alphavirus vector systems/platforms for human applications problematic.

A method of generating safe alphavirus vector platforms is disclosed herein, which can be used for the development of safer vaccines and/or therapies for animal and human use. In a preferred embodiment, approved safe alphavirus vaccines (live attenuated alphavirus) can be used in place of alphavirus for the development of vector platforms. In other words, while other researchers have used alphavirus replicons for the development of vector platforms, an improvement on this technique is to use proven safe attenuated alphavirus vaccines. In a most preferred embodiment, the TC-83 live attenuated vaccine, which has been proven safe in humans, can serve as a basis for making a vector platform that can be used to make vaccines and therapeutic gene vectors.

As used herein, vaccines are defined as a molecule, compound or composition, which when administered to a human or animal produces an immune response in the human or animal. Such an immune response can be useful to prevent, or lessen the probability of, infection by a pathogen, or to remove, reduce, or slow the progression of a pathogen or condition. In this regard, the skilled practitioner realizes that a vaccine need not perfectly and permanently prevent infection nor must a vaccine treatment provide a complete and perfect cure in order to provide a useful and desirable result. Likewise, therapies and therapeutic molecules are understood to mean treatments and compositions, which when introduced into a person or animal provide a desired benefit, which may include alleviation of an undesirable condition and/or a slowing of the progression or cure for a disease, but need not provide a complete and perfect cure or complete alleviation of a condition in order to be useful.

The TC-83 vaccine has been previously developed (Kinney et al., 1993). The TC-83 vaccine is the only live attenuated vaccine approved in the U.S. to date as an Investigational New Drug for vaccination of people against infections with VEE virus. The TC-83 vaccine has been safely administered to approximately 8,000 people in the U.S. In 80% of vaccine recipients, no adverse symptoms to TC-83 vaccine were reported. In the remaining 20% of recipients, only mild symptoms such as headaches have been observed. Interestingly, in approximately 20% of TC-83 vaccine recipients, or “non-responders”, no antibody to TC-83 antigens was elicited. Although these individuals needed additional vaccinations, for the vaccine vector, this is an additional beneficial feature because hypothetically, no self-immunity to the vector particles will be elicited in such non-responders. In such persons, repeated administrations of the same vector platform carrying various vaccines or/and therapies, with no self-immunity is possible.

In addition to the improved safety for administration into people, the TC-83 vaccine also offers safety advantages in vaccine manufacturing. TC-83 is the only strain of VEE approved for the use at the biosafety level-2 containment. Use of the TC-83 vaccine strain as a basis for vector platform development and manufacturing will not require BSL-3 containment. This can drastically reduce both risks and costs associated with the manufacturing of vaccines and/or therapies based on alphavirus vectors.

TC-83 based vectors combine the safety aspects of a proven safe vaccine with the advantageous vector features of the VEE virus. TC-83 comprises multiple, independently attenuating mutations (Table I; Kinney et al., 1993). Since TC-83 has been derived from VEE virues, the vector platforms derived from TC-83 can have the safety of a vaccine as well as the efficacy of the VEE vector, for example the ability to stimulate innate immune response, preferential targeting of the professional antigen-presenting cells in vivo, absence of pre-existing immunity, and high levels of expression of heterologous genes (Pushko et al., 1997; 2000; 2001; U.S. Pat. No. 5,792,462).

Briefly, the vector platforms and systems described herein can provide advantages over prior platforms including but not limited to: Vector platform is derived from human vaccine rather than a human pathogen. Vectors contain vector RNA of the vaccine VEE genotype rather than wild-type VEE genotype. Attenuating mutations in both nonstructural and structural regions rather than mutations in structural region only. Comprises up to about 17 attenuating mutations compared to the wild-type VEE. Multiple attenuating mutations will be retained even in the event of a recombination between a replicon and a helper nucleotide molecule. TC-83 VEE vaccine genotype a long history of safe use in humans. Replicon vector non-structural region contains an attenuating mutation. Can be handled at BSL-2 containment rather than BSL-3 containment. Vaccination of research and manufacturing personnel is not required.

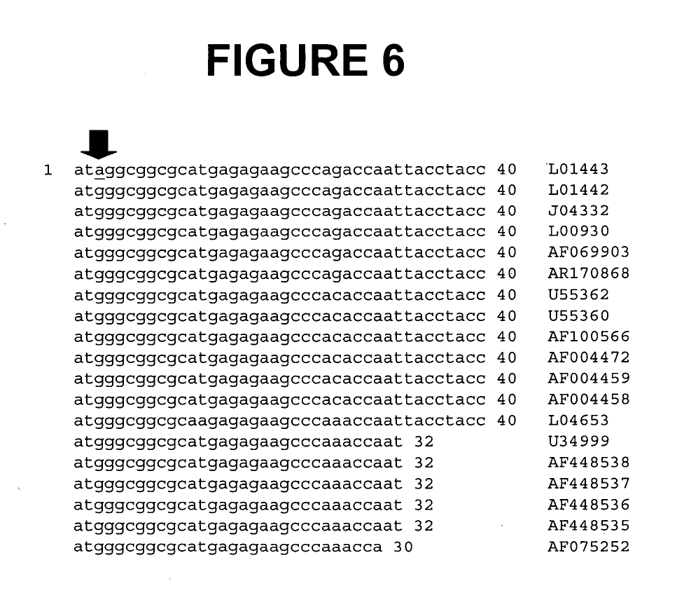

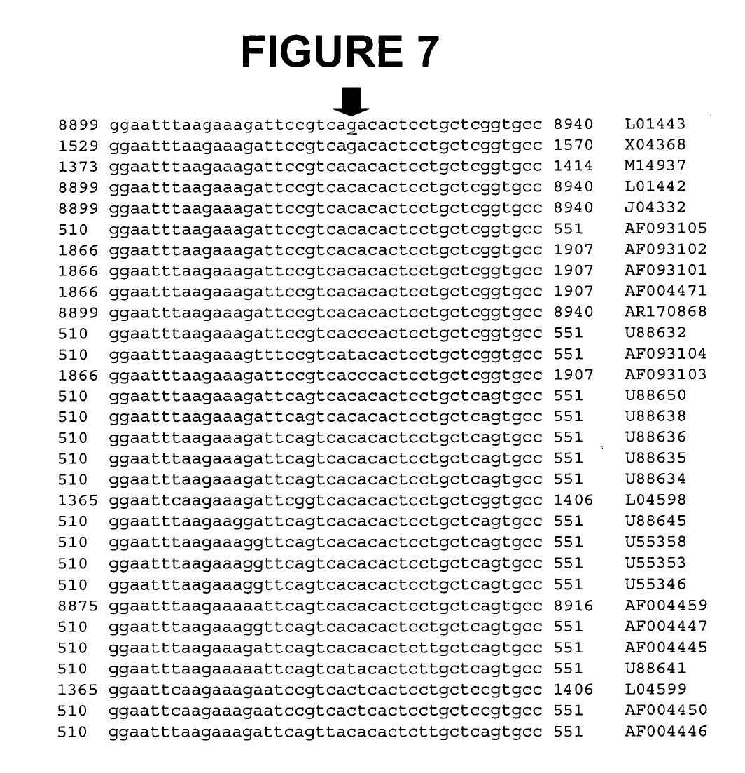

Human live attenuated alphavirus vaccines other than TC-83 can also be used for the development of safe alphavirus vector platforms. In this regard, FIGS. 6 and 7 demonstrate that sequences between strains of VEE are highly conserved so that one of ordinary skill in the art can readily identify locations in any VEE strand corresponding to attenuating mutations found in TC-83. By comparing sequences it will be possible to identify corresponding locations in other alphaviruses so as to make safe attenuated virus vector platforms by reference to the disclosure and examples described herein. Incorporation of 2, 3, 4, 5, 6, 7, 8, 9, 10, 11, 12, 13, 14,15, 16, 17 or more attenuating mutations identified by comparison of the TC-83 and other VEE genome sequences into a vector can improve the safety of the vector.

In addition, several alphavirus virus vaccines are being developed, or undergoing clinical trials, or being used as vaccines for Venezuelan, Eastern, and Western encephalitis viruses. For example, strain V3526 of VEE is undergoing Phase I clinical trials as a potential new live attenuated vaccine for VEE virus. Strain 3526 of VEE is also being considered for downgrading to BSL-2 biocontainment. V3526 only contains mutations in the structural region. So, if a structural region from V3526, or the mutations found therein, is used in a vector platform, it may be desirable to also use a TC-83 non-structural region or incorporate mutations found in the TC-83 non-structural region into the vector. Yet, another live attenuated VEE virus human vaccine is being used for vaccination of biomedical personnel at risk in Russia. Therefore, in addition to live attenuated, human TC-83 vaccine, other live attenuated alphavirus vaccines can also be used as a basis for the development of safe alphavirus vector platforms for the delivery of vaccines and therapies in people in accordance with the teaching and examples provided herein.

New vector platforms and methods of using live attenuated human alphavirus vaccines including the TC-83 human vaccine for VEE virus are disclosed herein. In a preferred embodiment, the nucleic acid molecules and vector platforms described herein can be based on the genome of live attenuated human TC-83 vaccine. Nucleic acid replicons, vectors, helpers, and plasmids as wells as complete vector platform systems can be generated from the genome and/or the nucleotide sequence of the TC-83 VEE vaccine virus.

Total RNA from the TC-83 vaccine virus can be isolated using phenol extraction or a similar method. TC-83 RNA can be used as a template for generating cDNA fragments corresponding to the replicon and/or helper RNAs, by using reverse transcription and polymerase chain reaction (RT-PCR) method. Replicon and helper cDNAs can be generated by RT-PCR using oligonucleotide primers designed using the known sequence of VEE and/or TC-83 so that a cDNA copy of any desired region of TC-83 RNA corresponding to the replicon or/and helper RNA is generated. Alternatively, DNA fragments encoding the TC-83 replicon and helper molecules can be synthesized directly using biochemical or/and chemical methods of DNA synthesis. Mutations present in the TC-83 genome, or another attenuated virus genome, can be introduced into regions derived from wild type virus by site specific mutagenesis or a similar method.

Any combination of RT-PCR, chemical DNA synthesis, site-specific mutagenesis, and/or other methods can be used to generate DNA fragments encoding a replicon and/or helper nucleic acid molecules, which are derived from, or contain mutations present in, the TC-83 vaccine or another VEE or live attenuated alphavirus vaccine.

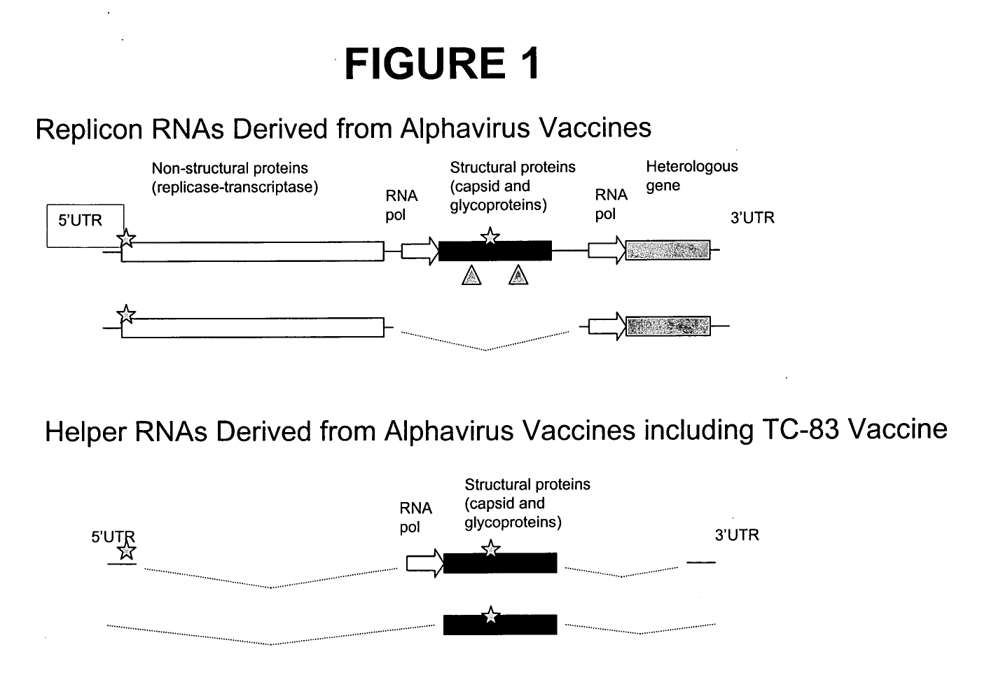

DNA fragments corresponding to the TC-83 replicon and helper molecules can be introduced into permissive recombinant DNA cloning systems downstream from functional RNA polymerase promoters. The insertion of heterologous gene(s) into the replicon or other modifications can be performed in the resulting cloned replicon and helper DNAs. The desired replicon and helper DNA or RNA molecules can be generated, for example as depicted in FIG. 1, using standard molecular biology cloning methods. RNA molecules can be made using any appropriate method, including transcription in vitro and/or in vivo, for example in eukaryotic cells containing the cDNA molecules.

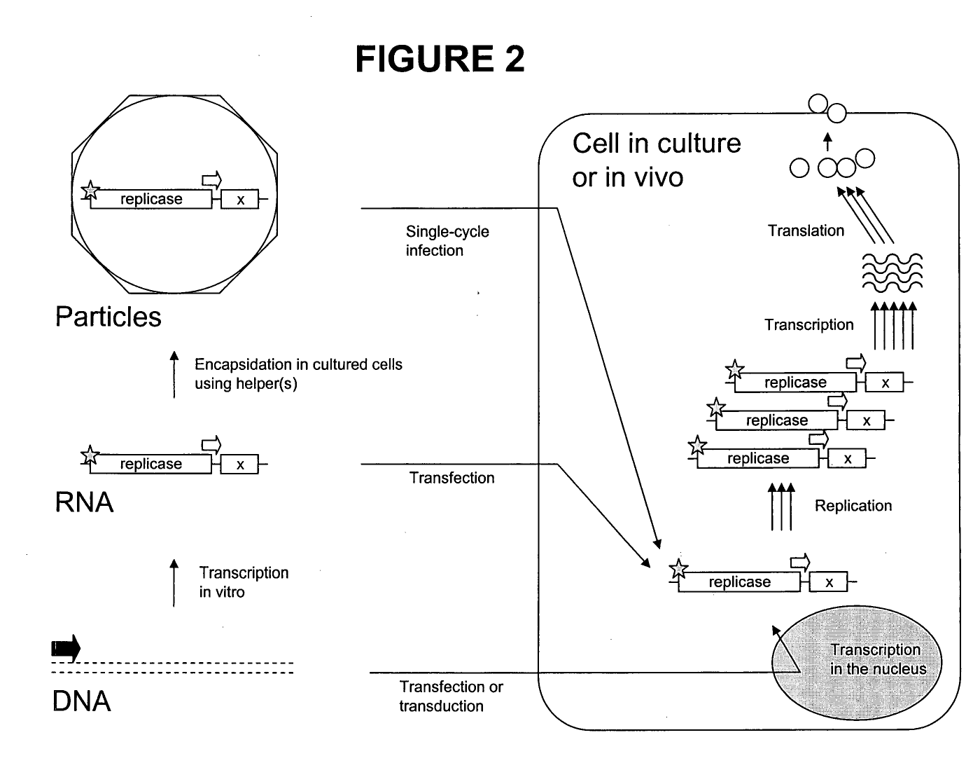

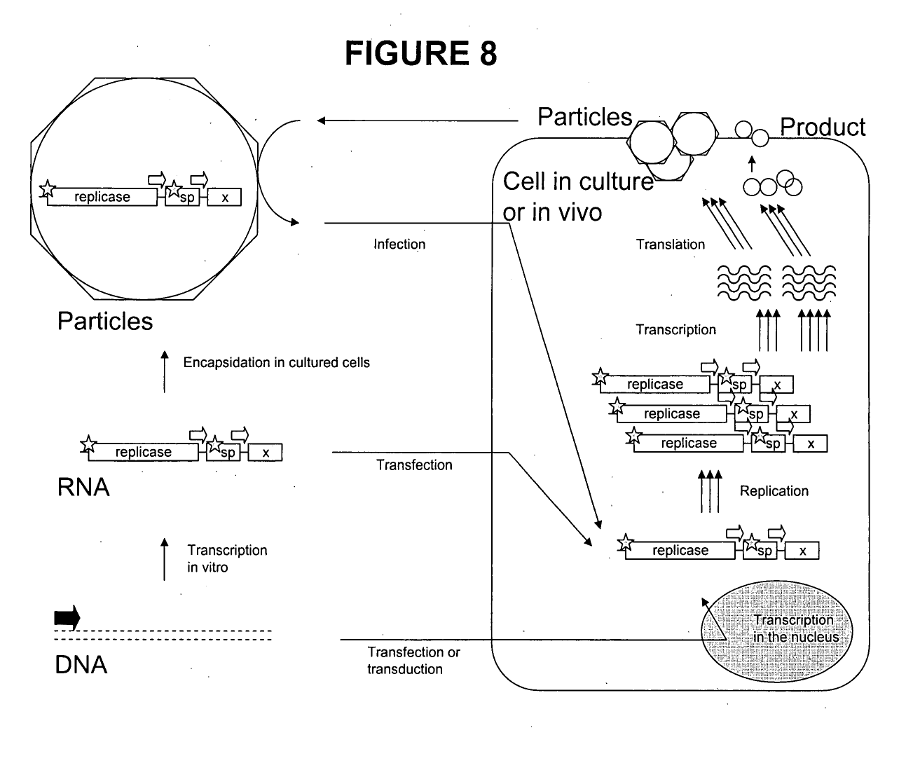

In one embodiment, plasmids containing the cDNAs for the TC-83 replicon and/or helper downstream from a functional in vitro RNA polymerase promoter are used as a template for in vitro transcription. If desired, transcription reactions can be carried out in the presence of the linearized plasmids as templates, or/and in the presence of cap analogue, or/and other means for improving quantity and/or quality of transcription reactions. RNA molecules can be recovered. The RNA molecules can then be transferred into eukaryotic cells in vitro or/and in vivo (FIGS. 2, 8).

Alternatively, the TC-83 replicon and/or helper RNAs can be generated directly in eukaryotic cells in culture or/and in vivo directly from the DNA molecules, which encode the replicon and helper RNAs downstream from a functional RNA polymerase promoter (FIGS. 2, 8). Such DNAs encoding replicon and/or helper sequences are introduced into the cells as described below. In the cells, DNAs function either in an episomal form or may be integrated into the host genome, with or without the use of selective pressure. The transcription and accumulation of the RNA replicon and/or helper molecules can take place inside the cells.

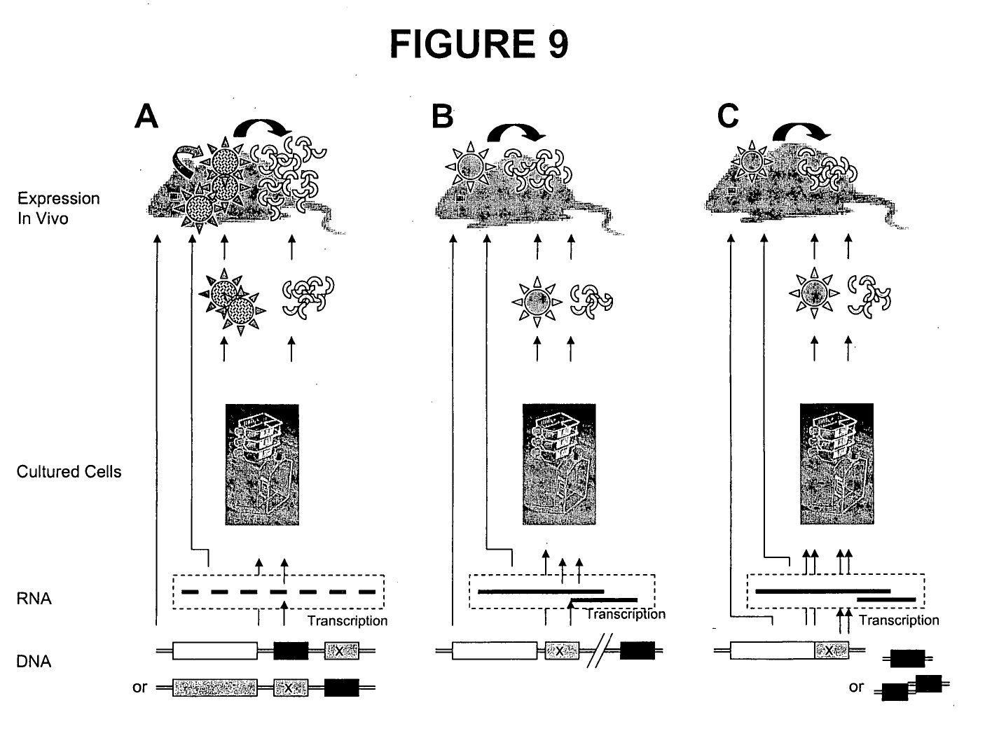

The RNA and/or DNA vectors (e.g., replicon molecules comprising heterologous genes) can be introduced into a permissive expression system, for example, eukaryotic cells in culture and/or by administration in vivo, for the purpose of expressing proteins or/and nucleic acids. Generally speaking, host cells as used herein are permissive cells. The expressed proteins and/or nucleic acids can provide a prophylactic, diagnostic and/or therapeutic effect (FIG. 2; FIG. 8; FIG. 9). That is to say, a gene to be expressed can be transferred into host cells by a “vector” (DNA, RNA, and/or replicon particles) that contains all necessary elements to ensure gene transfer and/or expression in cells. Such elements can include cell receptors, RNA polymerase promoters, enhancers, ribosome binding sites, transcription termination signals, polyadenilation signals.

A replicon RNA molecule as described herein represents a vector and/or a vector platform because it is capable of gene transfer and expression of a heteroldgous gene in a eukaryotic cell. The term “platform” indicates a vector that can be used for the transfer and expression of various heterologous genes, i.e. a vector capable of accepting a heterologous gene and can include any other system elements useful for production of a vector. RNA or DNA vector platforms can be used for the development of many human vaccines and therapeutics. The transfer of RNA or/and DNA molecules into host cells can be achieved using nucleic acid molecular transfer methods such as physico-chemical, biochemical, or/and biological methods including but not limited to, diffusion, electroporation, lipid- or ion-mediated transfection, virus transduction (Vasilakis et al., 2003), transfer of nucleic acids using TC-83 replicon particles, or/and other methods.

In one embodiment, TC-83 derived RNA replicon molecules, which can be generated in vitro and that encode a heterologous gene or genes are directly (with or without additives) used for transfection of cultured cells using calcium phosphate, liposomes, electroporation, and/or other methods. Alternatively, replicon RNA molecules can be introduced directly in vivo, for example by injection, as either “naked” RNA alone or RNA combined with the additives, which improve RNA stability, facilitate gene transfer, or/and have other beneficial characteristics (FIGS. 2, 8).

In alternative embodiments, illustrated for example in FIGS. 2 and 8, TC-83 derived DNA replicon molecules can be delivered into host cells. In this case, TC-83 replicon RNA molecules capable of driving expression of heterologous genes can be generated in the cells directly from the TC-83 DNA molecules thereby avoiding an in vitro transcription step. TC-83 derived DNA vectors encoding TC-83 derived replicon RNAs encoding heterologous genes are introduced into host cultured cells in vitro or directly in vivo, by using appropriate DNA transfer methods. The latter methods can include but are not limited to, injection of “naked” DNA with or without additives, or the use of other virus vectors for example vaccinia virus or/and adenovirus vectors to deliver DNA molecules encoding a TC-83 derived replicon into cultured cells or/and into hosts in vivo. For example, a DNA molecule encoding a TC-83 or other alphavirus derived replicon as described herein can be inserted into a “gutless” adenovirus vector and packaged into adenoviral particles using an appropriate adenoviral helper cell. These adenoviral particles can then be used to transfer the alphavirus replicon into host cells in vitro or in vivo. Transcription of the DNA in the host cells produces RNA replicons capable of propagation and optionally packaging as alphavirus particles and capable of driving expression of one or more heterologous genes.

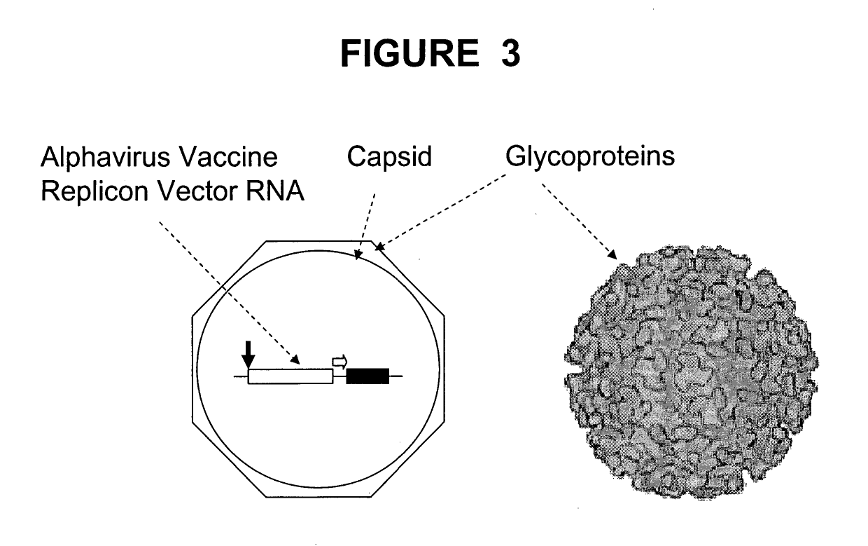

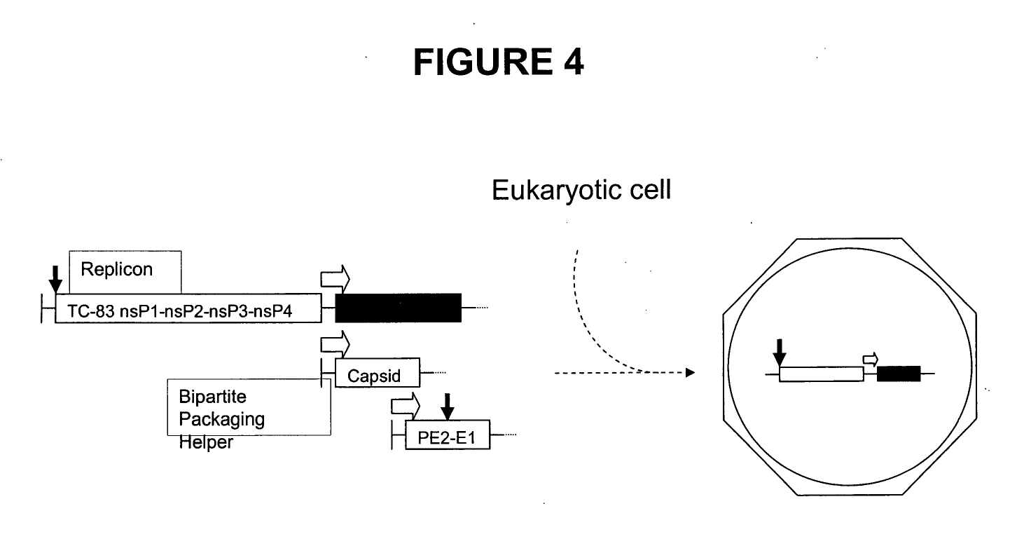

In another alternative embodiment, TC-83 derived RNA replicon molecules can be delivered into host cultured cells or into a host in vivo using TC-83 replicon particles (FIG. 2; FIG. 3; FIG. 8). The latter can be generated in vitro or in vivo by combining a TC-83 derived replicon RNA together with a TC-83 derived helper RNA molecule, which encodes the TC-83 structural proteins that can encapsidate the TC-83 derived replicon RNA molecules into replicon particles (FIG. 4).

Replicon particles have advantageous gene transfer capabilities. In order to generate replicon particles, replicon molecules encoding a heterologous product can be introduced into the host cells along with helper molecules encoding the TC-83 envelope and capsid proteins. In host cells, the TC-83 capsid and envelope proteins expressed from the helper molecule encapsidate the replicon RNA into the TC-83 replicon particles. Helper RNAs derived from other alphavirus vaccines or alphaviruses also can be used for the purpose of encapsidating TC-83 derived replicon RNAs, or another replicon RNA carrying at least one attenuating mutation, into replicon particles. Replicon particles can be produced without the assistance of helper nucleic acids in the case of a replicon that comprises a structural gene region, preferably including one or more attenuating mutations.

Replicon particles deliver replicon RNA into cells with higher efficiency compared to physico-chemical or biochemical methods such as “naked” RNA and/or DNA vector platforms. Replicon particles deliver replicon RNA into cells by using a specific receptor-mediated transfer mechanism. Up to 100% of cells exposed to replicon particle vectors can express heterologous product. Alphavirus-like replicon particles containing replicon RNA molecules having at least one attenuating mutation derived from the TC-83 vaccine (FIG. 3; FIG. 4) provide a safety feature. Previous alphavirus vectors derived from VEE, replicon RNA or replicon particles do not contain any attenuating mutations in any non-structural parts of the replicon (Pushko et al.,1997). In another embodiment, TC-83 derived replicon particles are generated directly in cultured cells in vitro or/and in vivo, by using DNA encoding both a TC-83 derived replicon and TC-83 helper RNAs, as illustrated in FIG. 8.

For example, a one or more DNA molecules encoding (i) a TC-83 derived replicon RNA comprising one or more heterologous genes and (ii) a TC-83 derived helper RNA encoding TC-83 structural proteins can be introduced into cultured cells or directly into a host in vivo using any appropriate DNA transfer methods. Transfer methods can include but are not limited to, injection of “naked” DNA as well as virus vectors for example vaccinia virus or/and adenovirus, which are used for delivery of TC-83 derived DNA molecules into host cultured cells or/and in vivo. Such platforms can provide that in the cells, new replicon particles can be generated together with the heterologous gene product. This results in infection of additional cells with the replicon, which is capable driving expression of the heterologous gene product in additional cells. If desired, product or/and replicon particles can be harvested from host cells.

In another example, DNA encoding a TC-83 derived replicon including a structural gene region is introduced into host cells in vitro or in vivo. The DNA is transcribed into a RNA replicon within the host cells that is capable of self replication and packaging into replication-competent particles. That is, a single administration of DNA can then generate a self propagating RNA vector, which due to its nature as a vaccine is safe. Alternatively, such a self propagating RNA can be generated in vitro by DNA-dependent RNA polymerase using a DNA encoding the replicon as a template for in vitro transcription as illustrated in FIGS. 8 and 9A. RNA can be also introduced into a host cell. In either case, the resulting host cell containing self replicating RNA vector is capable of generating the heterologous gene product from the heterologous gene from the RNA or DNA molecules. Along with the gene product, replicon particles are also generated from the RNA that has been transcribed from the DNA in the host cell.

Advantages of such a propagation-competent platform include that replicon particles produced in host cells can continue to infect additional cells, which can lead to a significant increase in the production of the gene product. Product or/and particles can be harvested from host cells. Replicon particles can then be used to infect host cells in vitro in order to generate more replicon particles, or/and to generate more heterologous gene product. Among other uses, heterologous gene product can be used to produce a vaccine and/or therapeutic effect. The product of the heterologous gene can be also isolated from cells and administered in vivo to produce a desired vaccination/therapeutic effect. Replicon particles containing such self replicating and packaging RNA, the RNA itself, or DNA encoding a replicon can be administered directly in vivo to animals or humans to provide a desired vaccine/therapy product directly in vivo as illustrated in FIGS. 8 and 9A.

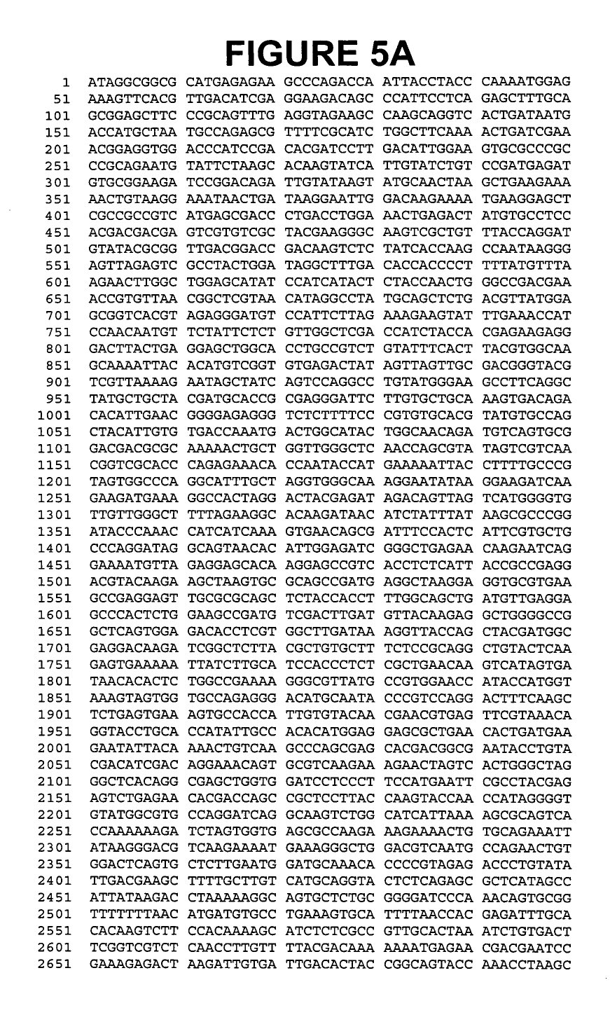

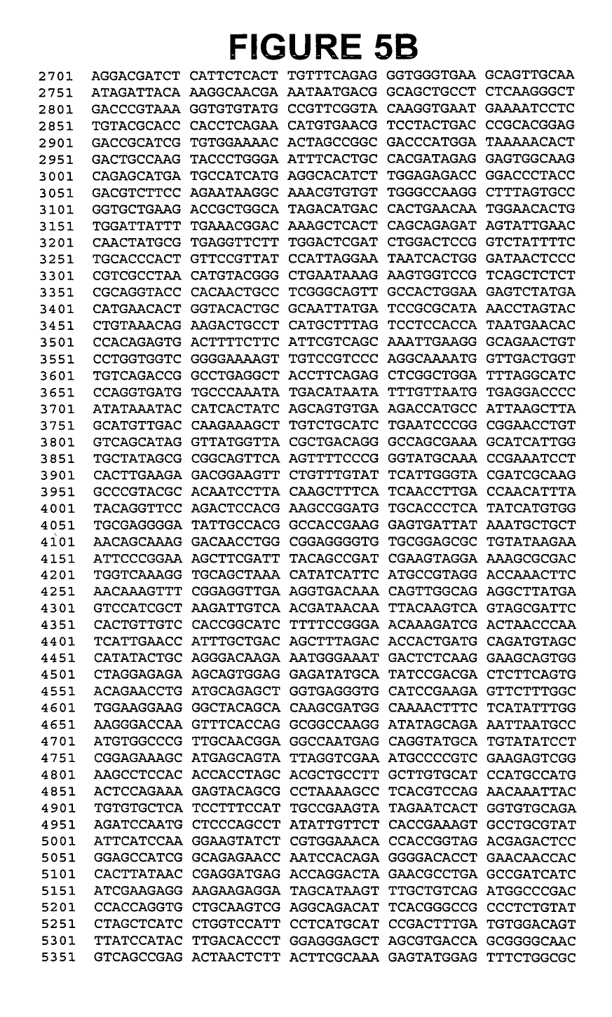

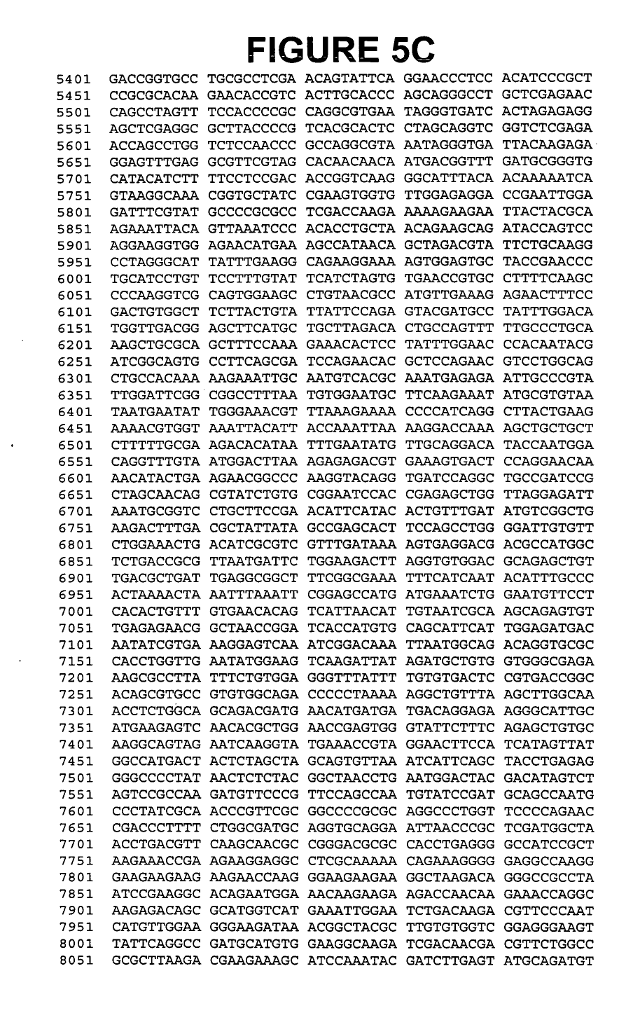

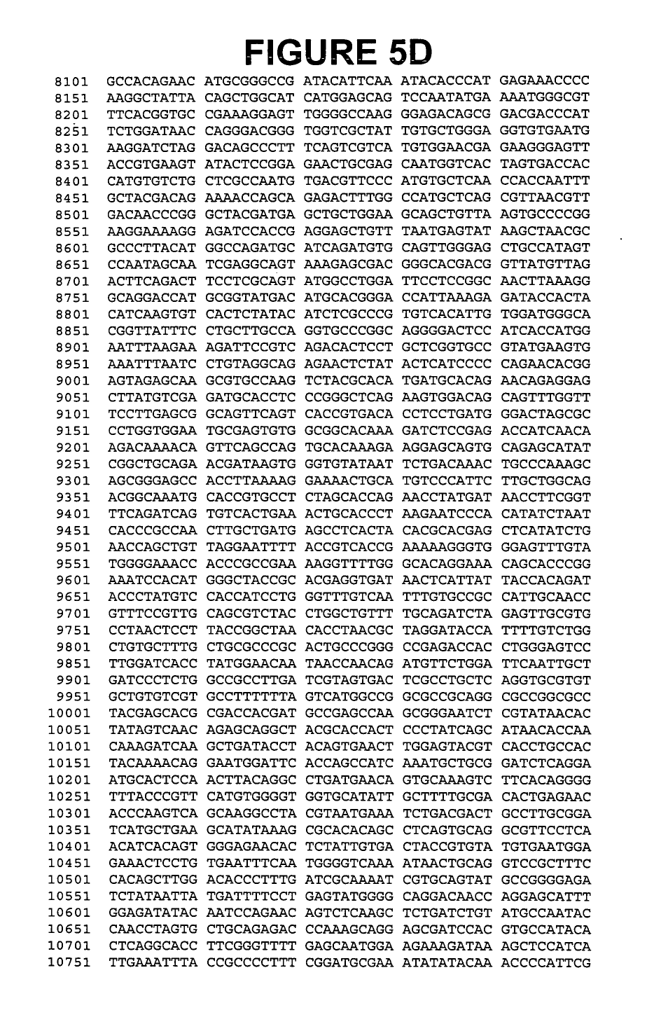

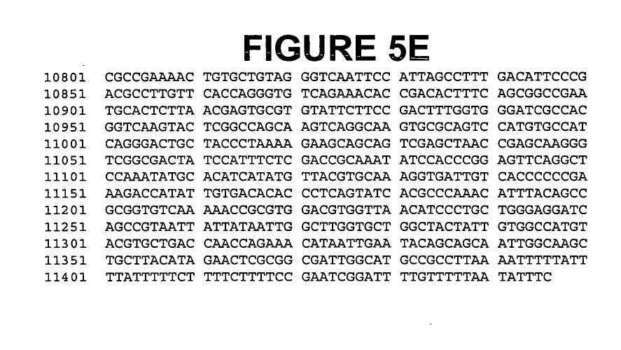

The vector platforms described herein can provide advantageous safety and efficacy characteristics as vaccines and/or therapeutics, because they are derived from the TC-83 vaccine or similarly safe attenuated alphavirus. The nucleotide sequence of the genome of TC-83 vaccine is depicted in FIG. 5. The TC-83 genome contains unique attenuating mutations in the 5′-UTR shown in FIG. 6 as well as in the structural gene region as shown in FIG. 7. An attenuating mutation in the 5′-UTR is present in TC-83 derived replicon particles as illustrated in FIG. 3, which can provide a significant improvement of existing alphavirus vectors as a new use for the TC-83 vaccine.

Replicon RNA molecules as described herein, when delivered by RNA, DNA, or/and replicon particles into cultured cells and/or host cells in vivo or in vitro, express a heterologous gene product in these cells in vitro or in vivo, which can be accumulated in the cells or secreted from the cells. Such gene product can be a beneficial nucleic acid or a polypeptide (for example, a gene product that prevents or treats diseases or induces immune response or other beneficial effects).

In one embodiment, such a heterologous nucleic acid or protein is used as a purified product isolated from cultured cells and then administered to a recipient. In an alternative, preferred embodiment, product is generated directly in vivo in the tissues of the recipient using a vector as described herein, for a beneficial effect. In this case, purification is avoided. Safety is ensured by the genotype of the alphavirus vaccine, which in a preferred embodiment, is derived from the TC-83 vaccine.

A heterologous gene, for example a vaccine-relevant or therapy-relevant gene or gene fragment, can be inserted into the cDNA of the TC-83 alphavirus downstream from the subgenomic 26S promoter. In order to facilitate insertion of a heterologous gene, a polylinker sequence containing recognition sites for restriction nucleases can be introduced downstream from the subgenomic 26S promoter. For insertion of a polylinker or/and of a heterologous DNA, the TC-83 cDNA nucleotide sequences located within, or in the vicinity of, the cleavage site for the Tth111 I restriction endonuclease (at nt 7544 in FIG. 5) can be used. After insertion of a heterologous gene in this manner, the genes encoding the TC-83 structural proteins are separated from the 26S promoter by the heterologous gene. In order to restore transcription and expression of the TC-83 structural proteins, a duplicate 26S promoter can be introduced. Alternatively, the structural protein genes and the duplicate 26S promoter can be introduced upstream from the heterologous gene and its 26S promoter as described below in the Example.

In other exemplary molecules, the TC-83 structural protein genes can be deleted. In this case, the structural proteins can be expressed from one or more helpers provided in trans. Preferably, deletion of the TC-83 structural proteins can be accomplished by deleting the fragment that encompasses the region from the 5′ (nt ˜7562) to the 3′ end of the structural polyprotein gene (nt 11329) of a TC-83 cDNA. For such a deletion, the Tth111 I site can be used (nt 7544) as well as Hpa I (nt 11229) or other restriction sites in the vicinity of nt 7562 and 11339 of the TC-83 genome. Of course, other methods also can be used for deletion or modification of the alphavirus genes, for example, polymerase chain reaction (PCR).

In addition to insertion of a heterologous gene, or/and deletion of the TC-83 structural protein genes, nucleotide sequences that are important for the functions of the vector, such as untranslated regions, promoters, etc., are preferably either preserved, or reconstituted from source material. For example, a second copy of the 26S promoter can be inserted in a vector that comprises both a heterologous gene region and a structural gene region. In the resulting propagation-competent nucleic acid molecule, one copy of the 26S promoter controls transcription of the heterologous gene(s), and the second copy of the 26S promoter controls transcription of the TC-83 structural proteins. The 26S promoter encompasses from approximately the Apa I site at nt 7505 to approximately the Tth111 I site at nt 7544 of the TC-83 cDNA sequence. Similarly, when the structural protein genes are deleted, the regions of the 26S promoter and/or of the 3′ untraslated region (UTR) are preferably preserved. The 26S promoter provides for the transcription of the downstream heterologous gene, whereas the integrity of the 3′ UTR is important for replication of the replicon molecules.

Heterologous genes can include therapeutic genes such as the following genes that can be therapeutic for treatment of cancer: prostate-specific antigen (PSA), Her2/neu, mucin, and the like. Vaccine genes can include Influenza HA, HPV L1, Ebola GP, HIV env, Lassa GP, and the like. Therapeutic genes may also encode RNA molecules that can affect expression of endogenous host genes, such as interfering RNAs, micro RNAs, ribozymes, and the like. Therapeutic genes may also encode peptides having desired binding properties such as antibodies and binding fragments, peptide aptamers, and the like. It will eb appreciated that the vector platforms described herein can be used generally to obtain expression of any heterologous gene or nucleic acid sequence in vivo or in vitro without limitation.

From the foregoing it will be appreciated that among the embodiments disclosed herein, at least the following preferred embodiments may be noted:
a. A “replicon” RNA molecule comprising 5′ untranslated region, nonstructural gene region, RNA-dependent RNA polymerase promoter region, structural gene region, a separate RNA polymerase promoter region, a heterologous nucleic acid sequence, and 3′ untranslated region, in which at least one of the above-mentioned regions derived from, or present in, the TC-83 vaccine or contains at least one mutation derived from, or present in, the TC-83 vaccine, or other live attenuated alphavirus vaccine (FIG. 1). In preferred embodiments, the replicon is a propagation-competent replicon.
b. A “replicon” RNA molecule according to embodiment (a), in which the structural gene region and one RNA polymerase promoter are partially or completely deleted (FIG. 1).
c. A “helper” RNA molecule according to embodiment (a), in which the nonstructural gene region and a RNA polymerase promoter with a heterologous nucleic acid sequence are partially or completely deleted (FIG. 1). A variant of this embodiment is a bipartite helper, in which the structural gene region is split between the two RNA helper molecules (FIG. 4).
d. A “helper” RNA molecule according to embodiment (a), in which the 5′ untranslated region, nonstructural gene region, RNA polymerase promoters, a heterologous nucleic acid sequence, and/or 3′ untranslated region are partially or completely deleted (FIG. 1).
e. A DNA molecule encoding a “replicon” RNA molecule according to embodiment (a) from a DNA-dependent RNA polymerase promoter. Preferably, the DNA molecule encodes a replication-competent replicon RNA molecule.
f. A DNA molecule encoding a “replicon” RNA molecule according to embodiment (b) from a DNA-dependent RNA polymerase promoter.
g. A DNA molecule encoding a “helper” RNA molecule according to embodiment (c) from a DNA-dependent RNA polymerase promoter.
h. A DNA molecule encoding a “helper” RNA molecule according to embodiment (d) from a DNA-dependent RNA polymerase promoter.
i. A DNA molecule encoding (1) a “replicon” RNA molecule according to embodiment b, and (2) a “helper” RNA molecule according to embodiment (c).
j. A DNA molecule encoding (1) a “replicon” RNA molecule according to embodiment b, and (2) a “helper” RNA molecule according to embodiment (d).
k. A eukaryotic cell containing at least one nucleic acid molecule according to embodiments (a) through (j).
l. A vector platform comprising a “replicon” RNA molecule according to embodiment (a) (FIG. 8). Preferably, the RNA molecule is replication-competent.
m. A vector platform comprising a “replicon” RNA molecule according to embodiment (a) encapsidated within a eukaryotic cell according to embodiment (k) into an alphavirus-like particle containing proteins encoded by the structural gene region within the said RNA molecule (FIG. 8). Preferably, the RNA molecule is replication-competent.
n. A vector platform comprising a “replicon” RNA molecule according to embodiment (b) (FIG. 2).
o. A vector platform comprising a “replicon” RNA molecule according to embodiment (b) encapsidated within a eukaryotic cell according to embodiment (k) into an alphavirus-like particle containing proteins encoded by the structural gene region within a “helper” RNA molecule according to embodiment (c) (FIG. 2, FIG. 4).
p. A propagation-defective vector platform comprising a “replicon” RNA molecule according to embodiment (b) encapsidated within a eukaryotic cell according to embodiment (k) into an alphavirus-like particle containing proteins encoded by the structural gene region within a “helper” RNA molecule according to embodiment (d) (FIG. 2).
q. A vector platform comprising a “replicon” RNA molecule according to embodiment (b) encapsidated within a eukaryotic cell according to embodiment (k) into an alphavirus-like particle containing proteins encoded by the structural gene region within a DNA molecule according to embodiment 7 (FIG. 2).
r. A vector platform comprising a “replicon” RNA molecule according to embodiment (b) encapsidated within a eukaryotic cell according to embodiment (k) into an alphavirus-like particle containing proteins encoded by the structural gene region within a DNA molecule according to embodiment 8 (FIG. 2).
s. A vector platform comprising a “replicon” RNA molecule according to embodiment (b) generated from a DNA molecule according to embodiment (i) and encapsidated within a eukaryotic cell according to embodiment (k) into an alphavirus-like particle containing proteins encoded by the structural gene region within a DNA molecule according to embodiment 9 (FIG. 8).
t. A vector platform comprising a “replicon” RNA molecule according to embodiment (b) generated from a DNA molecule according to embodiment (j) and encapsidated within a eukaryotic cell according to embodiment (k) into an alphavirus-like particle containing proteins encoded by the structural gene region within a DNA molecule according to embodiment (1) (FIG. 8).
u. A vector platform comprising a DNA molecule according to embodiment (e) (FIG. 8).
v. A vector platform comprising a DNA molecule according to embodiment (f) (FIG. 2).
x. A vector platform comprising a DNA molecule according to embodiment (g) (FIG. 8).
y. A vector platform comprising a DNA molecule according to embodiment (j) (FIG. 8).
z. Embodiments (a) through (y), in which at least one attenuating mutation located within the “replicon” molecule or within the alphavirus vector particle.

aa. Embodiments (a) through (z) in which in place of the TC-83 vaccine any live attenuated alphavirus vaccine is used including but not limited to, vaccines for Venezuelan equine encephalitis (or encephalomyelitis) virus, Semliki Forest virus, Sindbis virus, Eastern equine encephalitis virus, Western equine encephalitis virus.

TABLE II
Mutations within the genomic RNA of live attenuated vaccine strain
TC-83 as compared to wild type VEEV. At least two of these mutations
have been proven to be strongly attenuating (Kinney et al., 1993).

| Gene | nt # | VEEV* | | TC-83** | |
|---|---|---|---|---|---|
| 5′-UTR | nt 3 | G | | A | |
| nsP1 | nt 1007 | C | aa F | G | aa L |
| | nt 1533 | C | | G | |
| | nt 1534 | G | aa R | C | aa A |
| nsP3 | nt 4809 | T | aa S | A | aa T |
| E2 | nt 8584 | G | aa K | T | aa N |
| | nt 8816 | C | aa H | T | aa Y |
| | nt 8922 | C | aa T | G | aa R |
| | nt 9073 | A | aa P (silent) | G | |
| | nt 9138 | T | aa V | A | aa D |
| | nt 9279 | T | aa I | A | aa N |
| | nt 9450 | C | aa T | T | aa I |
| | nt 9487 | T | aa H (silent) | C | |
| | nt 9531 | G | aa G | A | aa E |
| | nt 10481 | T | aa L | A | aa I |
| E1 | nt 10633 | A | | T | |
| 3′-UTR | nt 11404 | T | | deleted | |

*From the cloned sequence of wild type VEEV, strain V3000
**From GenBank Accession No. L01443

As used herein, the term “or” includes “and” and singular forms include pluralities, unless clearly indicated otherwise. For example, a “platform or a system” includes embodiments that may be platforms and/or systems, and “a molecule” means one or more molecules.

While the invention has been described in detail with reference to preferred embodiments thereof, it will be apparent to one skilled in the art that various changes can be made, and equivalents employed, without departing from the scope of the invention. Further in this regard, the following examples are illustrative of aspects of the invention but should not be construed as limiting in any way.

### EXAMPLES

### Example 1

### Cloning and Production of Replicon and Helper RNAs

Total RNA is extracted from TC-83 vaccine using phenol extraction or a similar method. The cDNAs corresponding to TC-83 replicon and helper RNAs are derived by reverse transcription and polymerase chain reaction (RT-PCR) using extracted TC-83 viral RNA and the TC-83 sequence-specific oligonucleotide primers. Compared to wild type VEE virus, there are 17 mutations in the TC-83 genome (Table II).

The cDNA fragments corresponding to replicon and helper RNAs are cloned into plasmid containing functional DNA-dependent RNA polymerase so that in the result of transcription in vitro or in vivo, functional RNA replicon and/or helper are generated.

The heterologous gene is cloned downstream from the TC-83 replicase and 26S promoter in the transcription plasmid containing the TC-83 replicon cDNA (FIG. 1). An exogenous gene is either cloned in addition to the full-length TC-83 genome, or substituted for the genes that encode for the capsid and two envelope glycoproteins. RNA replicons as described herein may comprise exogenous gene either upstream from the structural protein genes (e.g., as in FIGS. 1 and 8), or downstream from the structural protein genes. In the other replicon molecule (e.g., as in FIG. 1), the TC-83 virus RNA promoter sequence and the genes that encode for the TC-83 replicase are left intact, whereas structural proteins are deleted. When the replicon is introduced into cultured cells, the heterologous gene is expressed.

When plasmid DNA contains replicon or/and helper RNA downstream from functional in vitro phage promoter, for example T7 promoter, the replicon and helper RNAs are prepared by in vitro transcription of the recombinant plasmids using T7 RNA polymerase, for example RiboMAX Large Scale RNA Production System (Promega, Inc., Madison, Wis.).

Alternatively, plasmid DNA containing replicon or/and helper RNA downstream from functional eukaryotic promoter, such as cytomegalovirus promoter/enhancer, serve as a template for synthesis of replicon and helper RNA molecules in cultured cells or in vivo.

### Example 2

### Protein Expression and Production of Alphavirus Replicon Particles

Eukaryotic cells are transfected by electroporation and incubated for approximately 30 hr. In order to demonstrate expression of heterologous protein from the replicon or TC-83 proteins from the helpers, cells are lysed in a buffer containing 50 mM Tris-HCl (pH 6.8), 5% 2-mercaptoethanol, 10% glycerol, and 1% sodium dodecyl sulfate. Proteins are separated, for example using 7% or 4 to 12% polyacrylamide gels. Western blotting is carried out using serum recognizing heterologous protein, or a TC-83 vaccine-specific serum, or monoclonal antibodies, followed by the appropriate peroxidase labeled secondary antibodies.

The TC-83 replicon particles are prepared by cotransfecting eukaryotic cells, for example Chinese hamster ovary (CHO) cells. For example, CHO cells can be transfected with replicon RNA along with the TC-83 c-helper and gp-helper RNAs (FIG. 4). As an example, eukaryotic cells are cotransfected by electroporation (0.4-cm gap cuvette; three pulses, 0.85 kV, 25 mF) with replicon RNA and helper RNAs. Particles are isolated from culture supernatants. Culture supernatants are clarified by centrifugation at 4000 times gravity for 10 min, and particles are concentrated and partially purified by pelleting at 28,000 rpm for 5 h in an SW28 rotor through 20% (wt/wt) sucrose in phosphate-buffered saline (pH 7.4).

### Example 3

### Titration of Particles

Titers are determined by immunofluorescence assay (IFA). CHO or BHK cells are grown to subconfluency in eight-well chamber slides, and particles are diluted at 10-fold increments in the EMEM containing 10% FBS. The diluted particles are absorbed (0.1 ml/well) onto CHO or BHK cell monolayers for 1 h at 37° C. Then, 0.3 ml of the medium is added per well and incubation is continued for about 16 h. Cells are fixed with cold acetone and probed with appropriate polyclonal or monoclonal antibodies specific for the heterologous gene product. Fluorescein-labeled secondary antibodies to immunoglobulin G (IgG) (heavy and light chains) are used at a 1:25 dilution. For double-staining IFA, a mixture of antigen-specific antibodies is used, followed by a mixture of rhodamine- and fluorescein-labeled secondary antibodies.

Cell nuclei are stained with 1 mg of 49,69-diamidino-2-phenylindole (DAPI) per ml in VectaShield mounting medium (Vector Labs, Inc., Burlingame, Calif.). The VRP titers are expressed as infectious units (IU).

### Example 4

### Immunizations

Particles are diluted in phosphate-buffered saline, pH 7.4. Laboratory animals, for example female Balb/c mice or guinea pigs are inoculated subcutaneously (s.c.) or intramuscularly (i.m.) at day 0 with a total of 0.5 ml containing 10⁷ infectious units (IU) of particles. Alternatively, animals are vaccinated by other RNA replicons as described herein and/or DNA molecules encoding such replicons. At 28-day intervals, two booster inoculations are administered.

### Example 5

### Serological Tests and Plaque Assays

Enzyme-linked immunosorbent assay (ELISA) is performed with purified protein as the substrate antigen. Sera from immunized animals is initially diluted 1:50 and then serially diluted 1:3, and a reaction stronger than the average reaction with negative control serum plus two standard deviations is considered positive.

For Western blotting, sera are pooled and assayed at 1:500 dilution.

Neutralizing antibodies are determined by 80% plaque reduction neutralization assay (PRNT80). Sera are initially diluted 1:10 and then serially diluted 1:2 in Hanks' balanced salt solution containing 10 mM HEPES and 10% guinea pig complement. Diluted serum (0.5 ml) is incubated with vaccine-relevant virus. For example, sera are incubated with target virus for 1 h at 37° C. in a total volume of 1 ml. Virus is absorbed on Vero cells in six-well plates (0.2 ml/well) for 1 h at 37° C., overlaid with 2 ml of 0.5% agarose in basal medium Eagle containing 10 mM HEPES and 5% FBS, and incubated for 4 days. A second overlay containing 5% neutral red is applied, plaques are counted 24 h later, and the serum dilution required to achieve 80% plaque reduction is determined. Neutralizing antibody for TC-83 vaccine virus is determined similarly, except that for incubation with TC-83 virus, serum is heat inactivated for 30 min at 56° C. and serially diluted 1:2 in Hanks' balanced salt solution containing 25 mM HEPES and 2% heat-inactivated FBS, and cells are incubated for 1 day before the second overlay. Similarly, PRNT is conducted for some other viruses, with slight modifications.

### Example 6

### Virus challenge

In order to demonstrate that vaccination with vectors and/or vector platforms described herein provides protection against infection with pathogens, challenge experiments are carried out. Challenge is carried out ˜28 days after the final dose of particles in mouse or guinea pig models. For example, guinea pigs are challenged s.c. or i.n. with lethal doses (LD₅₀) of target virus. The challenge virus is administered in a total volume of 0.5 ml in EMEM containing 2% FBS. Animals are observed daily for ˜31 days as described, and survival and changes in the appearance and behavior of the animals (mortality and morbidity) are recorded. Blood samples are taken on the days indicated after challenge and viremia levels were determined by plaque assay. Research is conducted in compliance with the Animal Welfare Act and other regulations relating to experiments involving animals.

### Example 7

### Influenza Vaccine Vector Construction

The cDNA corresponding to nt 1-7552 of TC-83 is generated using reverse transcription and polymerase chain reaction (RT-PCR) with primers 5′-GAT CGA TTA ATA CGA CTC ACT ATA GAT AGG CGG CGC ATG AGA GAA GC-3′ and 5′-GTC GCG ATA CGC GTT TTC GAA TGG CGC GCC TGA TAT CTA GAC TAT GCC GCA TTC GAA AAC GCG TAT CGC GA-3′. The resulting RT-PCR fragment is cloned into the pCR2.1-TOPO plasmid (Invitrogen). Then, the RT-PCR fragment corresponding to nt 1-7552 is subcloned into pcDNA3.1 plasmid downstream from the CMV promoter (Invitrogen). This results in pRM01 plasmid.

The following ApaI-NotI fragment is then cloned into the ApaI-NotI-digested pRM1 plasmid: 5′-GGGCCCCTAT AACTCTCTAC GGCTAACCTG AATGGACTAC GACATAGTCT AGCGATCGCG ATATCTTCGA ATAATTGAAT ACAGCAGCAA TTGGCAAGCT GCTTACATAG AACTCGCGGC GATTGGCATG CCGCCTTAAA ATTTTTATTT TATTTTTCTT TTCTTTTCCGA ATCGGATTTT GTTTTTAATAT TTCAAAAATC TAGACTCGAG CGGCCGC-3′. The resulting pRM02 plasmid contains the TC-83 sequence corresponding to (1) the 5′ untranslated region including attenuating mutation at nt 3 derived from the TC-83; (2) non-structural protein region, (3) 26S promoter, (4) multicloning site downstream from the 26S promoter, and (5) the 3′ untranslated region of the TC-83 sequence, whereas the TC-83 structural proteins are deleted. Multicloning site is then used for cloning of a heterologous gene derived from influenza virus.

The resulting pRM03 plasmid contains DNA molecule encoding a replicon. When the pRM03 DNA is transfected into the Chinese Hamster Ovary (CHO) or other susceptible cultured cells using Fugene 6 or a similar reagent, it generates a RNA replicon. Influenza hemagglutinin gene is expressed from this RNA (FIG. 10). Alternatively, pRM03 plasmid is injected in vivo, where it generates a RNA replicon. Thus, in the cells in vitro or in vivo, a RNA replicon is generated from the DNA.

### Example 7

### Influenza Vaccine Self-Packaging Vector Construction

Alternatively, the TC-83 structural gene region corresponding to nt 7501-11330 of the TC-83 sequence is cloned from the TC-83 vaccine by the RT-PCR using primers 5′- AAG GGC CCC TAT AAC TCT CTA CGG C-3′ and 5′ - AAG GGC CCC TCT CAA TTA TGT TTC TGG TTG GT-3′. The resulting fragment containing attenuating mutations is cloned into a pCR2.1-TOPO plasmid is subcloned as a ApaI-ApaI fragment into the ApaI site of pRM03 plasmid. This results in plasmid pRM04 that contains two 26S promoters. In the pRM04, the first 26S promoter is located upstream from the TC-83 structural proteins, whereas the second 26S promoter is located upstream from the influenza gene within the multicloning site. The resulting pRM04 plasmid contains the TC-83 sequence corresponding to (1) the 5′ untranslated region including attenuating mutation at nt 3 derived from the TC-83; (2) non-structural protein region, (3) 26S promoter, (4) the TC-83 structuralprotein region containing attenuating mutations, (5) second 26Spromoter, (6) heterologous influenza gene, and (7) the 3′ untranslated region of the TC-83 sequence. When the pRM04 DNA is transfected into the Chinese Hamster Ovary (CHO) or other susceptible cultured cells using Fugene 6 or a similar reagent, it generates an RNA replicon. This RNA directs expression of (1) the TC-83 structural proteins from the first 26S promoter, as well as (2) influenza gene from the second 26S promoter. Alternatively, pRM04 plasmid is injected in vivo, where it generates a RNA replicon. In the cells, the TC-83 structural proteins encapsidate the RNA replicon into TC-83-like vector particles. These vector particles infect other cells, in which this process repeats (FIGS. 8, 9). Resulting in a greater number of cells expressing influenza gene.

### REFERENCES

The following are referenced herein or may otherwise contribute to the understanding of this disclosure:

U.S. Pat. No.

6,632,640 Vaccine against Staphylococcus intoxication
U.S. Pat. No. 6,541,010 Alphavirus RNA replicon systems
U.S. Pat. No. 6,531,135 Alphavirus RNA replicon systems
U.S. Pat. No. 6,521,235 Alphavirus RNA replicon systems
U.S. Pat. No. 6,517,842 Marburg virus vaccines
U.S. Pat. No. 6,495,143 Botulinum neurotoxin vaccine
U.S. Pat. No. 6,296,854 Live attenuated Venezuelan Equine encephalitis vaccine
U.S. Pat. No. 6,156,558 Alphavirus RNA replicon systems
U.S. Pat. No. 5,792,462 Alphavirus RNA replicon systems
U.S. Pat. No. 6,376,236 Recombinant alphavirus particles
U.S. Pat. No. 5,843,723 Alphavirus vector constructs
U.S. Pat. No. 6,015,694 Method for stimulating an immune response utilizing recombinant alphavirus particles.
U.S. Pat. No. 5,739,026 DNA expression systems based on alphaviruses
U.S. Pat. No. 6,566,093 Alphavirus cDNA vectors
U.S. Pat. No. 6,770,479 Anthrax Vaccine
U.S. Pat. No. 6,426,196 Alphavirus structural protein expression cassettes
U.S. Pat. No. 6,770,283 DNA expression systems based on alphaviruses
   Published US patent applications
20040146859 Ebola virion proteins expressed from Venezuelan equine encephalitis (VEE) virus replicons
20040121466 Alphavirus RNA replicon systems
20040009183 Vaccine against Staphylococcus intoxication
20030232036 Alphavirus RNA replicon systems
20030152590 Marburg virus vaccines
20020164582 Ebola virion proteins expressed from Venezuelan equine encephalitis (VEE) virus replicons
20020034521 Botulinum neurotoxin vaccine
20010016199 Alphavirus RNA replicon systems
   Other References
Davis N L, Brown K W, Johnston R E. A viral vaccine vector that expresses foreign genes in lymph nodes and protects against mucosal challenge. J Virol. June 1996;70(6):3781-7.
Davis N L, Powell N, Greenwald G F, Willis L V, Johnson B J, Smith J F, Johnston R E. Attenuating mutations in the E2 glycoprotein gene of Venezuelan equine encephalitis virus: construction of single and multiple mutants in a full-length cDNA clone. Virology. July 1991;183(1):20-31.
Hevey M, Negley D, Pushko P, Smith J, Schmaljohn A. Marburg virus vaccines based upon alphavirus replicons protect guinea pigs and nonhuman primates. Virology. November 10, 1998 ;251(1):28-37.
Kinney R M, Chang G J, Tsuchiya K R, Sneider J M, Roehrig J T, Woodward T M, Trent D W. Attenuation of Venezuelan equine encephalitis virus strain TC-83 is encoded by the 5′-noncoding region and the E2 envelope glycoprotein. J Virol. March 1993;67(3):1269-77.
Lee J S, Pushko P, Parker M D, Dertzbaugh M T, Smith L A, Smith J F. Candidate vaccine against botulinum neurotoxin serotype A derived from a Venezuelan equine encephalitis virus vector system. Infect Immun. September 2001;69(9):5709-15.
Nelson E L, Prieto D, Alexander T G, Pushko P, Lofts L A, Rayner J O, Kamrud K I, Fralish B, Smith J F. Venezuelan equine encephalitis replicon immunization overcomes intrinsic tolerance and elicits effective anti-tumor immunity to the ‘self’ tumor-associated antigen, neu in a rat mammary tumor model. Breast Cancer Res Treat. December 2003;82(3):169-83.
Polo J M, Belli B A, Driver D A, Frolov I, Sherrill S, Hariharan M J, Townsend K, Perri S, Mento S J, Jolly D J, Chang S M, Schlesinger S, Dubensky T W Jr. Stable alphavirus packaging cell lines for Sindbis virus and Semliki Forest virus-derived vectors. Proc Natl Acad Sci U S A. Apr. 13, 1999;13;96(8):4598-603.
Pushko P, Bray M, Ludwig G V, Parker M, Schmaljohn A, Sanchez A, Jahrling P B, Smith J F. Recombinant RNA replicons derived from attenuated Venezuelan equine encephalitis virus protect guinea pigs and mice from Ebola hemorrhagic fever virus. Vaccine. August 2000 15;19(1):142-53.
Pushko P, Geisbert J, Parker M, Jahrling P, Smith J. Individual and bivalent vaccines based on alphavirus replicons protect guinea pigs against infection with Lassa and Ebola viruses. J Virol. December 2001;75(23):11677-85.
Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. December 1997 22;239(2):389-401.
Smerdou C, Liljestrom P. Non-viral amplification systems for gene transfer: vectors based on alphaviruses. Curr Opin Mol Ther. April 1999;1 (2):244-51.
Vasilakis N, Falvey D, Gangolli S S, Coleman J, Kowalski J, Udem S A, Zamb T J, Kovacs G R. Transfection-independent production of alphavirus replicon particles based on poxvirus expression vectors. Nat Biotechnol. August 2003;21(8):932-5.
Willems W R, Kaluza G, Boschek C B, Bauer H, Hager H, Schutz H J, Feistner H. Semliki forest virus: cause of a fatal case of human encephalitis. Science. Mar. 16, 1979 16;203(4385):1127-9.

What is claimed is:

1. A RNA molecule comprising an alphavirus 5′ untranslated region, an alphavirus non-structural gene region, and an alphavirus 3′ untranslated region, and further comprising a RNA-dependent RNA polymerase promoter region operably coupled to a heterologous nucleic acid sequence upstream of the 3′ untranslated region, wherein one or more attenuating mutations are present in one or more of the alphavirus regions.

2. A RNA molecule according to claim 1 that does not include an alphavirus structural gene region.

3. An RNA molecule according to claim 1, wherein the attenuating mutations are nucleotides that are present in the TC-83 VEE alphavirus vaccine (GenBank Accession No. L01443) that are not present in wild-type VEE.

4. An RNA molecule according to claim 1, wherein the mutations include the substitution of an adenosine (A) in the position corresponding to nucleotide 3 of the TC-83 VEE genome as described in GenBank Accession No. L01443.

5. A helper RNA molecule comprising an isolated RNA polymerase region operably coupled to an alphavirus structural gene region wherein one or more attenuating mutations present in the TC-83 structural gene region are present in said alphavirus structural gene region.

6. A helper RNA molecule according to claim 5, comprising an alphavirus genome from which the non-structural gene region has been deleted.

7. A DNA helper molecule encoding alphavirus structural proteins having one or more attenuating mutations present in the TC-83 structural gene region.

8. A host cell comprising a nucleotide sequence stably integrated into the cellular genome that encodes the proteins encoded by an alphavirus structural gene region and having one or more attenuating mutations present in the TC-83 genome.

9. A helper RNA molecule according to claim 5, having a guanidine (G) in the position corresponding to nucleotide 8922 of the TC-83 VEE genome as described in GenBank Accession No. L01443.

10. A RNA molecule comprising an alphavirus 5′ untranslated region, an alphavirus non-structural gene region, a first RNA-dependent RNA polymerase promoter region, an alphavirus structural gene region, and an alphavirus 3′ untranslated region from an alphavirus genome, wherein one or more attenuating mutations are present in one or more of these regions, and further comprising a RNA-dependent RNA polymerase promoter region operably coupled to a heterologous nucleic acid sequence upstream of the 3′ untranslated region.

11. An RNA molecule according to claim 10 having attenuating mutations or entire regions that are present in the TC-83 VEE alphavirus vaccine (GenBank Accession No. L01443).

12. An RNA according to claim 11, having an adenosine (A) in the position corresponding to nucleotide 3 of the TC-83 VEE genome as described in GenBank Accession No. L01443 and a guanidine (G) in the position corresponding to nucleotide 8922 of the TC-83 VEE genome as described in GenBank Accession No. L01443.

13. A system comprising a RNA molecule according to claim 1, and one or more helper molecules or a host cell comprising one or more nucleotide sequences encoding alphavirus structural proteins.

14. A system comprising a RNA molecule according to claim 3, and one or more helper nucleic acid molecules or a host cell comprising one or more nucleic acid sequences encoding alphavirus structural proteins having one or more mutations found in the structural region of the TC-83 VEE genome.

15. A system according to claim 14, comprising two or more helper molecules comprising nucleotide sequences encoding different alphavirus structural proteins.

16. A DNA molecule comprising a nucleotide sequence encoding an RNA molecule according to claim 1.

17. A DNA molecule comprising a nucleotide sequence encoding an RNA molecule according to claim 3.

18. A DNA molecule comprising a nucleotide sequence encoding an RNA molecule according to claim 4.

19. A DNA molecule comprising a nucleotide sequence encoding an RNA molecule according to claim 5.

20. A DNA molecule comprising a nucleotide sequence encoding an RNA molecule according to claim 10.

21. A DNA molecule comprising a nucleotide sequence encoding an RNA molecule according to claim 11.

22. A DNA molecule comprising a nucleotide sequence encoding an RNA molecule according to claim 12.

23. A DNA molecule comprising a first nucleotide sequence encoding a RNA molecule according to claim 1 and a second nucleotide sequence encoding A helper RNA molecule comprising an isolated RNA polymerase region operably coupled to an alphavirus structural gene region having one or more attenuating mutations present in the TC-83 structural gene region.

24. A DNA molecule comprising a first nucleotide sequence encoding a replicon RNA molecule according to claim 3 and a second nucleotide sequence encoding a helper RNA, said helper molecule comprising an isolated RNA polymerase region operably coupled to an alphavirus structural gene region having one or more attenuating mutations present in the TC-83 structural gene region.

25. A DNA molecule according to claim 24 further comprising two adenoviral ITR sequences and an adenoviral encapsidation region.

26. An adenoviral particle comprising a DNA molecule according to claim 25.

27. An alphavirus particle comprising an RNA molecule according to claim 1.

28. An alphavirus particle comprising an RNA molecule according to claim 3.

29. An alphavirus particle comprising an RNA molecule according to claim 4.

30. An alphavirus particle comprising a replication-competent RNA molecule according to claim 10.

31. An alphavirus particle comprising a replication-competent RNA molecule according to claim 11.

32. An alphavirus particle comprising a replication-competent RNA molecule according to claim 12.

33. A method of making vector particles comprising:
introducing a RNA according to claim 1 or a DNA encoding said RNA into host cells;
wherein if said RNA or DNA does not comprise one or more operable nucleotides sequences encoding all alphavirus structural proteins necessary for particle formation then said host cells separately comprise one or more nucleotide sequences encoding the necessary alphavirus structural proteins, and,
recovering vector particles.

34. A method of making vector particles according to claim 33, wherein said RNA or DNA is introduced into host cells in a host organism.

35. A method of making vector particles according to claim 34, wherein said DNA encoding said RNA further separately comprises one ore more nucleotide sequences encoding alphavirus structural proteins.

36. A method of making vector particles according to claim 33, wherein said RNA or DNA is introduced into host cells in culture media.

37. A method of making vector particles comprising:
introducing a RNA according to claim 10 or a DNA encoding said RNA into host cells; and,
recovering vector particles.

38. A method of transferring a heterologous nucleotide sequence into a host cell comprising transferring a RNA molecule according to any one of claims 1-4 into the host cells.

39. A method of transferring a heterologous nucleotide sequence into a host cell comprising transferring a DNA molecule encoding an RNA molecule according to any one of claims 1-4 into the host cells.

40. The method of claim 38, further comprising transferring a helper nucleic acid molecule into said cell.

41. The method of claim 39 further comprising transferring a helper nucleic acid molecule into said cell.

42. The method of claim 39, wherein said DNA molecule further separately encodes alphavirus structural proteins.

43. A method of transferring a heterologous nucleotide sequence into a host cell comprising transferring a RNA molecule according to any one of claims 10-12 into the host cells.

44. A method of transferring a heterologous nucleotide sequence into a host cell comprising transferring a DNA molecule encoding an RNA molecule according to any one of claims 10-12 into the host cells.


SEQUENCE LISTING (55 sequences)

SEQ ID NO: 1 - DNA, 11446 bp, Venezuelan equine encephalitis virus
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttc

SEQ ID NO: 2 - DNA, 40 bp, Venezuelan equine encephalitis virus
ataggcggcgcatgagagaagcccagaccaattacctacc

SEQ ID NO: 3 - DNA, 40 bp, Venezuelan equine encephalitis virus
atgggcggcgcatgagagaagcccagaccaattacctacc

SEQ ID NO: 4 - DNA, 40 bp, Venezuelan equine encephalitis virus; identical to SEQ ID NO: 3

SEQ ID NO: 5 - DNA, 40 bp, Venezuelan equine encephalitis virus; identical to SEQ ID NO: 3

SEQ ID NO: 6 - DNA, 40 bp, Venezuelan equine encephalitis virus; identical to SEQ ID NO: 3

SEQ ID NO: 7 - DNA, 40 bp, Venezuelan equine encephalitis virus; identical to SEQ ID NO: 3

SEQ ID NO: 8 - DNA, 40 bp, Venezuelan equine encephalitis virus
atgggcggcgcatgagagaagcccacaccaattacctacc

SEQ ID NO: 9 - DNA, 40 bp, Venezuelan equine encephalitis virus; identical to SEQ ID NO: 8

SEQ ID NO: 10 - DNA, 40 bp, Venezuelan equine encephalitis virus; identical to SEQ ID NO: 8

SEQ ID NO: 11 - DNA, 40 bp, Venezuelan equine encephalitis virus; identical to SEQ ID NO: 8

SEQ ID NO: 12 - DNA, 40 bp, Venezuelan equine encephalitis virus; identical to SEQ ID NO: 8

SEQ ID NO: 13 - DNA, 40 bp, Venezuelan equine encephalitis virus; identical to SEQ ID NO: 8

SEQ ID NO: 14 - DNA, 40 bp, Venezuelan equine encephalitis virus
atgggcggcgcaagagagaagcccaaaccaattacctacc

SEQ ID NO: 15 - DNA, 32 bp, Venezuelan equine encephalitis virus
atgggcggcgcatgagagaagcccaaaccaat

SEQ ID NO: 16 - DNA, 32 bp, Venezuelan equine encephalitis virus; identical to SEQ ID NO: 15

SEQ ID NO: 17 - DNA, 32 bp, Venezuelan equine encephalitis virus; identical to SEQ ID NO: 15

SEQ ID NO: 18 - DNA, 32 bp, Venezuelan equine encephalitis virus; identical to SEQ ID NO: 15

SEQ ID NO: 19 - DNA, 32 bp, Venezuelan equine encephalitis virus; identical to SEQ ID NO: 15

SEQ ID NO: 20 - DNA, 30 bp, Venezuelan equine encephalitis virus
atgggcggcgcatgagagaagcccaaacca

SEQ ID NO: 21 - DNA, 42 bp, Venezuelan equine encephalitis virus
ggaatttaagaaagattccgtcagacactcctgctcggtgcc

SEQ ID NO: 22 - DNA, 42 bp, Venezuelan equine encephalitis virus; identical to SEQ ID NO: 21

SEQ ID NO: 23 - DNA, 42 bp, Venezuelan equine encephalitis virus
ggaatttaagaaagattccgtcacacactcctgctcggtgcc

SEQ ID NO: 24 - DNA, 42 bp, Venezuelan equine encephalitis virus; identical to SEQ ID NO: 23

SEQ ID NO: 25 - DNA, 42 bp, Venezuelan equine encephalitis virus; identical to SEQ ID NO: 23

SEQ ID NO: 26 - DNA, 42 bp, Venezuelan equine encephalitis virus; identical to SEQ ID NO: 23

SEQ ID NO: 27 - DNA, 42 bp, Venezuelan equine encephalitis virus; identical to SEQ ID NO: 23

SEQ ID NO: 28 - DNA, 42 bp, Venezuelan equine encephalitis virus; identical to SEQ ID NO: 23

SEQ ID NO: 29 - DNA, 42 bp, Venezuelan equine encephalitis virus; identical to SEQ ID NO: 23

SEQ ID NO: 30 - DNA, 42 bp, Venezuelan equine encephalitis virus; identical to SEQ ID NO: 23

SEQ ID NO: 31 - DNA, 42 bp, Venezuelan equine encephalitis virus
ggaatttaagaaagattccgtcacccactcctgctcggtgcc

SEQ ID NO: 32 - DNA, 42 bp, Venezuelan equine encephalitis virus
ggaatttaagaaagtttccgtcatacactcctgctcggtgcc

SEQ ID NO: 33 - DNA, 42 bp, Venezuelan equine encephalitis virus; identical to SEQ ID NO: 31

SEQ ID NO: 34 - DNA, 42 bp, Venezuelan equine encephalitis virus
ggaatttaagaaagattcagtcacacactcctgctcagtgcc

SEQ ID NO: 35 - DNA, 42 bp, Venezuelan equine encephalitis virus; identical to SEQ ID NO: 34

SEQ ID NO: 36 - DNA, 42 bp, Venezuelan equine encephalitis virus; identical to SEQ ID NO: 34

SEQ ID NO: 37 - DNA, 42 bp, Venezuelan equine encephalitis virus; identical to SEQ ID NO: 34

SEQ ID NO: 38 - DNA, 42 bp, Venezuelan equine encephalitis virus; identical to SEQ ID NO: 34

SEQ ID NO: 39 - DNA, 42 bp, Venezuelan equine encephalitis virus
ggaattcaagaaagattcggtcacacactcctgctcggtgcc

SEQ ID NO: 40 - DNA, 42 bp, Venezuelan equine encephalitis virus
ggaatttaagaaggattcagtcacacactcctgctcagtgcc

SEQ ID NO: 41 - DNA, 42 bp, Venezuelan equine encephalitis virus
ggaatttaagaaaggttcagtcacacactcctgctcagtgcc

SEQ ID NO: 42 - DNA, 42 bp, Venezuelan equine encephalitis virus; identical to SEQ ID NO: 41

SEQ ID NO: 43 - DNA, 42 bp, Venezuelan equine encephalitis virus; identical to SEQ ID NO: 41

SEQ ID NO: 44 - DNA, 42 bp, Venezuelan equine encephalitis virus
ggaatttaagaaaaattcagtcacacactcctgctcagtgcc

SEQ ID NO: 45 - DNA, 42 bp, Venezuelan equine encephalitis virus; identical to SEQ ID NO: 41

SEQ ID NO: 46 - DNA, 42 bp, Venezuelan equine encephalitis virus
ggaatttaagaaagattcagtcacacactcttgctcagtgcc

SEQ ID NO: 47 - DNA, 42 bp, Venezuelan equine encephalitis virus
ggaatttaagaaaaattcagtcatacactcttgctcagtgcc

SEQ ID NO: 48 - DNA, 42 bp, Venezuelan equine encephalitis virus
ggaattcaagaaagaatccgtcactcactcctgctccgtgcc

SEQ ID NO: 49 - DNA, 42 bp, Venezuelan equine encephalitis virus; identical to SEQ ID NO: 48

SEQ ID NO: 50 - DNA, 42 bp, Venezuelan equine encephalitis virus
ggaatttaagaaagattcagttacacactcttgctcagtgcc

SEQ ID NO: 51 - DNA, 47 bp, Artificial Sequence
  Description of Artificial Sequence Synthetic primer
gatcgattaatacgactcactatagataggcggcgcatgagagaagc

SEQ ID NO: 52 - DNA, 71 bp, Artificial Sequence
  Description of Artificial Sequence Synthetic primer
gtcgcgatacgcgttttcgaatggcgcgcctgatatctagactatgccgcattcgaaaacgcgtatcgcga

SEQ ID NO: 53 - DNA, 219 bp, Artificial Sequence
  Description of Artificial Sequence Synthetic pRM1 plasmid
gggcccctataactctctacggctaacctgaatggactacgacatagtctagcgatcgcgatatcttcgaataattgaatacagcagcaattggcaagctgcttacatagaactcgcggcgattggcatgccgccttaaaatttttattttatttttcttttcttttccgaatcggattttgtttttaatatttcaaaaatctagactcgagcggccgc

SEQ ID NO: 54 - DNA, 25 bp, Artificial Sequence
  Description of Artificial Sequence Synthetic primer
aagggcccctataactctctacggc

SEQ ID NO: 55 - DNA, 32 bp, Artificial Sequence
  Description of Artificial Sequence Synthetic primer
aagggcccctctcaattatgtttctggttggt